(19) United States
(12) Patent Application Publication
(10) Pub. No.: US 2010/0304380 A1
(43) Pub. Date: Dec. 2, 2010

(54) Quantitative DNA Methylation Imaging of Cells and Tissues

(75) Inventors: Jian TAJBAKHSH, Encino, CA (US); Daniel L. FARKAS, Los Angeles, CA (US)

Correspondence Address: Jian Tajbakhsh, 17128 Escalon Drive, Encino, CA, 91436, US

(73) Assignee: EPILUMINA, INC., Encino, CA (US)

(21) Appl. No.: 12/691,296

(22) Filed: Apr. 6, 2010

Related U.S. Application Data
(60) Provisional application No. 61/205,573, filed on Jan. 22, 2009.

(51) Int. Cl. C12Q 1/68

(52) U.S. Cl. 435/6

(57) ABSTRACT

The invention is a method using immunofluorescence, high-resolution imaging, and 3D image analysis to quantify spatial distribution of methylcytosine (MeC) in global DNA in individual cell nuclei, for the characterization of cells and tissues based on their nuclear MeC distribution patterns. The invented method is intended for basic research, clinical diagnostics and prognostics, pharmacology and toxicology, and molecular and cell-based therapy.

We describe an image-cytometrical tool performing rapid cell-by-cell assay to analyze the impact of environmental conditions on global DNA methylation, including alterations of methylcytosine load and the spatial reorganization of methylated DNA in cell nuclei. The assay can be combined with molecular approaches for profiling of cells and their genomes. In contrast to molecular methods, it provides information regarding the location of MeC in cell nuclei. The resulting assay can analyze thousands of cells in parallel in a cost-effective manner—currently not possible with molecular methods.

### CROSS-REFERENCE TO RELATED APPLICATIONS

This is a utility application based on the provisional U.S. patent application Ser. No. 61/205,573 filed Jan. 22, 2009, the entire contents of which are incorporated herein.

### FIELD OF THE INVENTION

The invention is related to a method using (i) immunofluorescence, (ii) high-resolution image acquisition, and (iii) 3D image analysis to extract and quantify the spatial distribution of methylcytosine (MeC) in global DNA (colocalization of methylcytosine and global DNA) in individual cell nuclei, particularly for the characterization of cells and tissues based on their nuclear MeC distribution patterns (FIG. 1). The invented method is intended to serve a variety of applications in basic research, clinical diagnostics and prognostics, pharmacology and toxicology, and molecular and cell-based therapy.

### BACKGROUND OF THE INVENTION

DNA methylation is an epigenetic mechanism, which plays a key role in cellular differentiation. Aberrant global methylation patterns are associated with complex diseases including several cancer types. In cancer the resulting epigenetic alterations on the DNA level fall into two categories: (i) gene-specific hypermethylation of CpG-dinucleotides in gene promoters in gene-rich genomic regions termed CpG-islands, and (ii) genome-wide hypomethylation, a large percentage of which occurs in repetitive DNA elements [1]. Molecular tools exist today to perform high-resolution DNA methylation profiling of cells and tumors. However, these methods are very challenged in terms of sensitivity and specificity, when it comes to the analysis of the methylation status of repetitive elements in single cells for two reasons: 1) it is currently impossible to identify the genomic location of most of the repetitive elements because of the high degree of similarity between their sequences, which generally makes the accurate sequencing of this type of DNA very difficult; 2) DNA of single cells is of very low copy number, usually below the sensitivity limit of molecular methods including sequencing. Additionally, the methylation profiling of large numbers of single cells to assess tumor inhomogeneity can become very costly and impractical. Therefore new approaches are necessary to characterize cells and tissues in a high-throughput cell-by-cell fashion, as non-epithelial cells usually infiltrate tumor tissue and may obscure important molecular information of cancer cells.

Given the prevalence and load of DNA methylation imbalances, especially hypomethylation of repetitive elements, cellular imaging of global nuclear DNA patterns may provide a powerful tool to characterize mammalian cells during differentiation and in their status of health versus disease, as the underlying molecular processes involve large-scale chromatin reorganization, visible by light microscopy. Therefore, nondestructive imaging-based cell/tissue assessments remain as indispensable tools in the pathologic diagnostics of solid tissue cancers. Unfortunately, current cell-based analyses in cancer diagnostics are mostly restricted to cell/tissue morphology and basic global DNA content measurement. However, there is more differential information embedded within the nuclear 3D structure of chromatin, which can be extracted by high-resolution imaging and image analysis for the characterization of cells and tissues in the combat against complex traits such as cancer.

We have developed Quantitative DNA Methylation Imaging (qDMI), an imaging-based tool for differential global epigenetic profiling of cells and tissues in situ [2]. QDMI is a nondestructive method that entails the parallel quantitative measurement of cytosine methylation in various potential nucleic acid markers, including single gene loci, and global DNA, in particular its heterochromatic portion, in conjunction with their topological distribution. This new method is based on image-cytometry by which the comparative spatial nuclear distribution (colocalization) of methylated cytosine and the different classes of DNA can be quantitated in the cells' nuclei.

Pharmaceutical drugs act on cellular processes or pathways to induce physiological changes. In addition drugs may also exert epigenetic effects, which affect gene expression and related chromatin architecture in cells. These latter effects can be targeted but also occur as unwanted adverse occurrences (side-effects) that might put cells, organs, and eventually the whole organism at risk [3]. Our rapidly accumulating knowledge of such possible changes in the current therapeutic practice calls for strategies and systems biology approaches in drug screening and drug discovery that can detect unwanted epigenetic effects, in order to improve the agents' clinical utility. These new approaches, which have been termed pharmacoepigenomics or toxicoepigenomics (in analogy to pharmaco-/toxicogenomics), if introduced into the drug development program, might also significantly reduce attrition in the early phases of drug development [3].

DNA and histones are the basic components of a chromosome, in which the DNA helix is wrapped around core histones to form the beads-on-a-string structure that is then folded into higher-order chromatin. Epigenetic changes, such as DNA methylation and histone modifications, are key regulators of gene expression and chromatin architecture in a cell- and tissue-specific manner. DNA methylation is considered the most robust among the different types of chromatin modifications, and researchers are now comfortable with the theory that DNA methylation can act as a causal factor in transcriptional inactivation. Because DNA methylation plays an important role in controlling cellular gene-expression programs, the DNA methylation machinery is an attractive pharmacological target [4]. Drugs that cause demethylation and the reactivation of ectopically methylated genes might be superior to many other therapeutic approaches that are targeted at specific functions of individual proteins.

Nevertheless it is important to recognize that hypermethylation of single gene promoters occurs against a strong background of general DNA hypomethylation including a decrease in the methylation load of heterochromatic regions of the genome. The loss of methyl groups is achieved mainly by hypomethylation of heterochromatin-residing repetitive DNA sequences, including transposable elements such as long interspersed nuclear elements (LINE), especially LINE-1 as part of the facultative heterochromatin, and satellite 2 (Sat2) DNA as part of the constitutive heterochromatin [5]. The extent of genome-wide hypomethylation converges closely with the degree of malignancy, in a tumor type-dependent manner. In normal cells, the activity and interaction of these classes of DNA with neighboring chromatin regions (in the nuclear space) are strongly suppressed by methylation and compaction through histone modifications. Therefore, there are multiple risks associated with an increase in demethylation of repetitive elements, which can lead to their decondensation and related consequences—Sat2 DNA hypomethylation may favor pericentromeric instability, and LINE-1 elements may become transcriptionally active and relocate into other parts of the genome causing enhanced genome instability through mutational effects such as silencing of tumor-suppressor genes or the activation of oncogenes. This is in fact a critical issue, as malignant cells already contain 20-60% less genomic methylcytosine than their normal counterpart.

Despite the extraordinary degree of complexity in chromatin modifications and signaling, which makes the identification of suitable compounds challenging, there is substantial enthusiasm for the development and implementation of epigenetic therapies in several human disease areas, especially oncology. Chromatin phenotype is known to be significantly disrupted in cancer. This has been demonstrated in many morphologic studies on cancer by the application of digital texture analysis for the quantitative analysis of chromatin phenotype in neoplasia—supporting the role of chromatin phenotype as a biomarker for diagnosis and prognosis, and thus emphasizing the potential of quantitative imaging as a powerful tool to characterize mammalian cells based on chromatin texture such as spatial nuclear methylcytosine distribution and colocalization patterns. Cellular imaging can be defined as the use of a system/technology capable of visualizing a cell population, single cell or subcellular structures, applied in a combination with image-analysis tools.

Since heterochromatin decondensation bears a serious risk in epigenetic therapy, it is important to consider the testing of new compounds and drugs using cell-based assays in the pre-clinical phases of drug discovery. Logically, these dedicated assays need to aim at the quantitative characterization of global DNA methylation changes in a high-throughput statistical fashion to verify unwanted effects such as increased hypomethylation of repetitive elements and its causal risk of heterochromatin decondensation, which can cause severe adverse reactions of cells. QDMI, recently developed by us, can measure two important parameters of DNA methylation changes: (I) the methylcytosine (MeC) load, and (II) the spatial nuclear codistribution of MeC and global DNA (gDNA). We have demonstrated that the codistribution patterns of these two classes of DNA can be utilized as signatures for the characterization of stem cells in differentiation and cancer cells upon treatment with demethylating agents. Using our novel algorithm we succeeded in tracking changes of higher-order DNA organization due to drug-induced demethylation on a genomic scale in mammalian tumour cells.

Demethylating agents cause structural reorganization of the genome in cell nuclei, as they not only alter the DNA methylation load but also influence its spatial distribution [2,6]. In particular, heterochromatin decondensation, as a secondary effect of global demethylation, results in the relocation of heterochromatic sites within the nucleus (which is associated with genome destabilization). As a consequence of these conformational and organizational changes of the DAPI-positive nuclear sites, the same DAPI signal intensity is spread out over a higher number of voxels. Thus, both MeC and DAPI have dynamic patterns in the cell nucleus that become more discernable in a joint 2D plots than in a 1D MeC plot, or even when the two signals are separately displayed in one dimension [7]. Kullback-Leibler's divergence is a valuable measure for quantitating dissimilarities within a cell population and this measure can be applied to any multi-color cellular assay that utilizes topological information of intracellular structures to assess cellular behavior [8]. The image analysis system can flexibly interrogate the cells with any number of targets and large amounts of 3D image stacks for high-content analysis. QDMI connects fluorescence techniques including immunocytochemistry and fluorescence in situ hybridization (FISH) to computational techniques for image analysis and data interpretation. Our experimental results underline the robustness of the method, but also its flexibility in dealing with a high dynamic range in sample size suitable for low to high-throughput cell-based screening [7]. The system is amenable to scale and can be implemented for biomedical research as well as the high-volume industrial routine.

Approximately 55% of the human genome is comprised of heterochromatin. Epigenetic drugs with demethylating effects have been shown to alter genome organization within mammalian cell nuclei [6]. Therefore, we used the genome and its 3D organization as an indicator to evaluate the effect of demethylating drugs on cancer cells in situ. Our approach provides a statistical measurement on the two classes of DNAs (MeC and DAPI-positive gDNA) as nuclear targets. The algorithm compares the relative distribution of signals derived from these two targets (from two color-channels), projects them onto scatter plots (FIGS. 3 and 4), and then measures the degree of similarities between the plotted signal distributions of cells within a population. This method offers a way to evaluate cellular response to external factors such as drugs and changes in culture conditions via dissimilarity assessment of relevant cellular structures. The algorithm developed combines the three major tasks: (1) automated segmentation of nuclei in a cell population, (2) subsequent nuclear pattern extraction, and (3) distance-based statistical measurement of cell similarity using Kullback-Leibler (K-L) divergence. This method considers the strength of statistical evaluation of intra-nuclear MeC/DAPI patterns, especially valuable when cell population homogeneity is difficult to be assessed due to lack of standardized reference and sample size.

U.S. Provisional Patent Application publication 61/205,573, entire contents of which are incorporated herein, discloses a concept for an imaging-based tool for differential global epigenetic profiling of cells and tissues in situ [2,3]. This tool combines the technologies of (i) immunofluorescence, (ii) high-resolution image acquisition, and (iii) 3D image analysis. This new method is based on image-cytometry by which the comparative spatial nuclear distribution (colocalization) of methylated DNA and heterochromatic DNA can be quantitated, subcellularly. It uses a commercially available specific antibody against methylated cytosine (MeC, the only nucleic acid base methylated in mammals) and 4′,6-diamidino-2-phenylindole (DAPI), respectively. The invented method is considered to serve a variety of applications, (a) in basic research, and (b) in clinical diagnostics and molecular and cell-based therapy. Differential Nuclear DNA Methylation Imaging targets two major fields that can immediately profit from this development: (1) the search for factors that contribute to pluripotency and cell fate in human embryonic stem cell expansion and differentiation, and (2) the characterization of tumor cells with regard to their heterogeneity in molecular composition and behavior, including in response to drugs.

### SUMMARY OF THE INVENTION

In accordance with preferred embodiments of the present invention, a concept of an image-cytometrical tool is provided for performing a variety of cell-based and tissue-based assays and analyses that utilize the three-dimensional nuclear distribution patterns of DNA methylation (methylcytosine).

Another object of this invention is to provide a rapid cell-by-cell assay that can analyze the impact of environmental conditions on global DNA methylation within cells, including alterations of methylcytosine load and the spatial reorganization of methylated DNA in cell nuclei. The assay can be used modular to molecular approaches in the profiling of cells and their genomes regarding their methylcytosine composition. In contrast to molecular methods quantitative DNA Methylation Imaging provides information regarding the location of methylated DNA in cell nuclei.

Another object of this invention is to provide an assay that can perform analysis of thousands of cells in parallel in a cost-effective manner, which is currently not possible with molecular methods.

### BRIEF DESCRIPTION OF THE DRAWINGS

FIG. 1 is a workflow of Quantitative DNA Methylation (MeC) Imaging comprised of three steps: (1) immunofluorescence with antibodies against MeC (and against a cell-type or cell-characteristic marker) and DNA counterstaining with DAPI, (2) high-resolution fluorescence microscopy to collect 2D optical sections of the cells, and (3) 3D fluorescence image analysis. The latter step leads to the output of scatter plots that display the distribution of MeC versus DAPI signals. This differential co-distribution pattern serves as a signature of the interrogated cells.

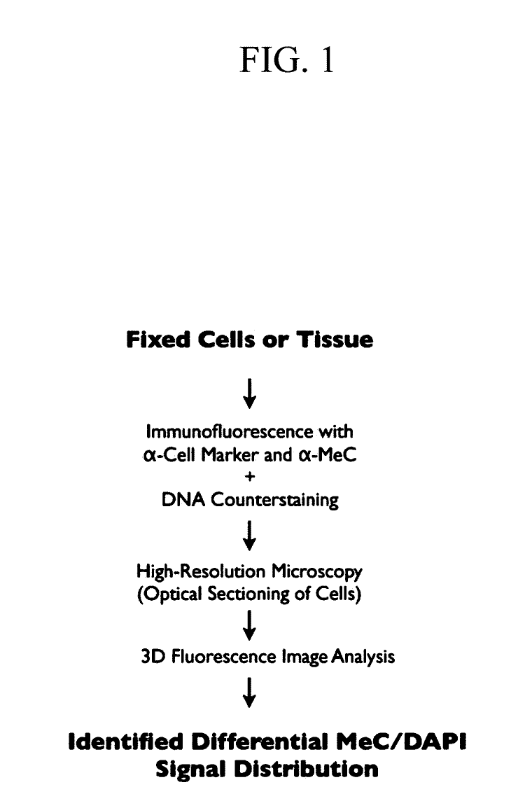

FIG. 2 shows a Test Microarray (included in the invention) for assessing the specificity of the anti-MeC antibody, which is crucial for the developed Differential Nuclear DNA Methylation Imaging method. The array is comprised of a glass microscopy slide coated with strepatvidin, onto which an array of two types of synthesized 24-mer oligonucleotides are deposited. The two oligos have the exact same length and nucleotides sequence. The only difference is that one of them contains two methylated cytosine molecules incorporated by phosphoramadite chemistry. The fluorescence in the image shows the specificity of the anti-MeC antibody used.

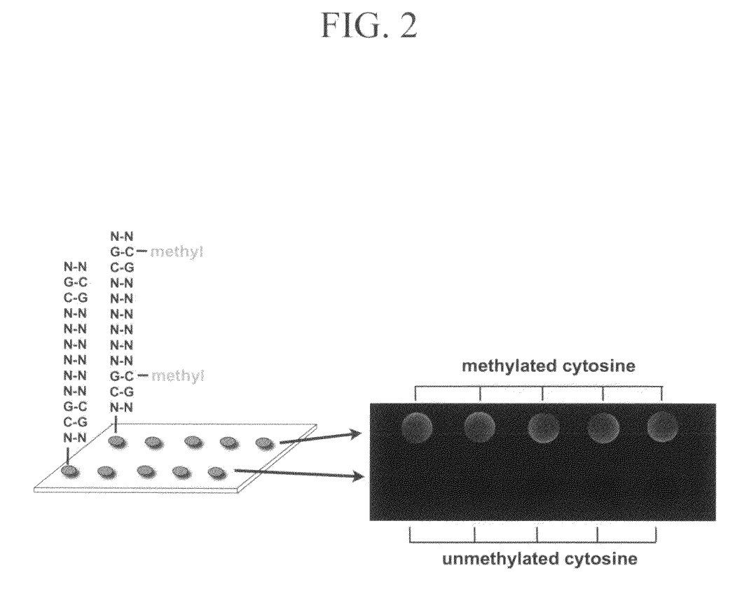

FIG. 3 shows a change of nuclear DNA methylation patterns that can be assessed by Quantitative DNA Methylation (MeC) Imaging. Upper row: untreated AtT20 mouse pituitary tumor cells in culture show a tight colocalization of MeC signals (green) and heterochromatin/DAPI (red), which can be seen in the overlay image as well as in the colocalization diagram. Lower row: in contrast, 5-azacytidine treated AtT20 cells show large foci that are uniquely comprised of three types of signals/genomic loci: hypermethylated regions distinct from heterochromatin (upper group), methylated regions that co-localize with heterochromatin (middle group), and hypomethylated regions of the heterochromatin (lower group).

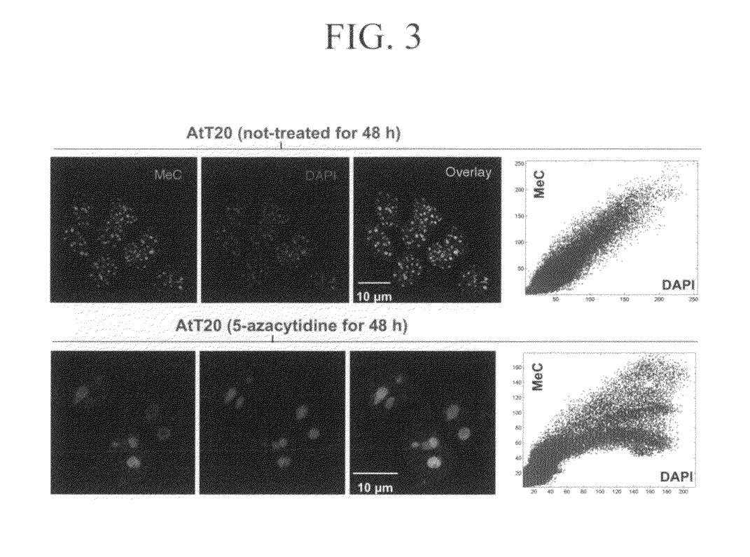

FIG. 4 shows an immunofluorescence image of TtT-GF mouse folliculostellate pituitary cell nuclei in culture show diverse response upon treatment with demethylating agent octreotide (100 nM) for 24 hours. The cell population was interrogated by Quantitative DNA Methylation Imaging: selected cells (numbered 1-5) were analyzed for colocalization of MeC signals (green) and heterochromatin/DAPI (blue), and results displayed as corresponding image-cytometry diagrams (right): cell 1 and 2 show less demethylation, whereas cells 3-5 display a stronger response (demethylation) to octreotide.

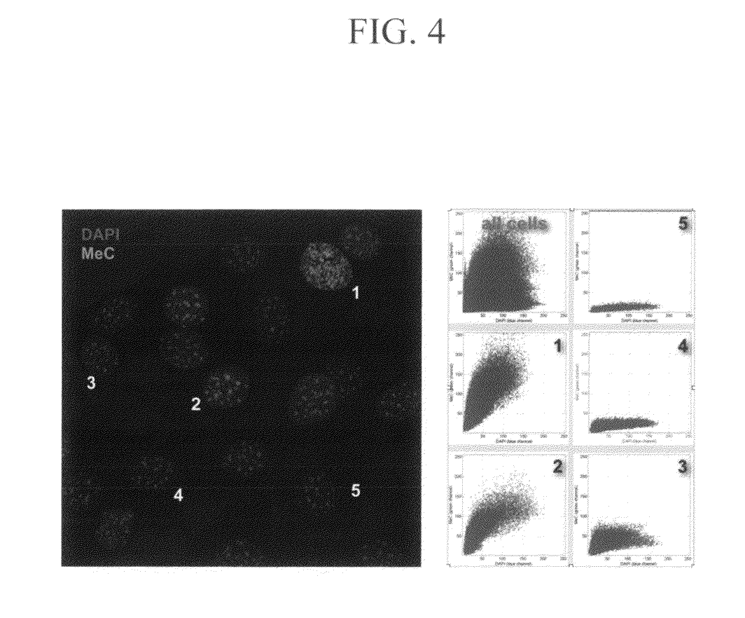

### REFERENCES

[1] Jones P A, Baylin S B (2007). The epigenomics of cancer. Cell 128, 683-692.
[2] Tajbakhsh J, Wawrowsky K A, Gertych A, Bar-Nur O, Vishnevsky E, Lindsley E H, Farkas D L (2008). Characterization of tumor cells and stem cells by differential nuclear methylation imaging. In: Farkas D L, Nicolau D V, Leif R C. Editors. Imaging, Manipulation, and Analysis of Biomolecules, Cells, and Tissues San Jose: Proceedings of the SPIE Vol.; 6859F1-10.
[3] Csoka A B, Szyf M (2009). Epigenetic side-effects of common pharmaceuticals: a potential new field in medicine and pharmacology. Med Hypotheses 73, 770-780.
[4] Yoo C B, Jones P A (2006). Epigenetic therapy of cancer: past, present and future. Nat Rev Drug Discov 5, 37-50.
[5] Ehrlich M (2005). DNA Methylation and Cancer-associated Genetic Instability. Chapter 3.6 in: Genome Instability in Cancer Development, Nathan Back, Irun R. Cohen, David Kritchevsky, Abel Lajtha, Rodolfo Paoletti and Erich A. Nigg, Editors, ISSN 0065-2598.
[6] Haaf T (1995). The effects of 5-azacytidine and 5-azadeoxycytidine on chromosome structure and function: implications for methylation-associated cellular processes. Pharmacol Ther 65, 19-46.
[7] Gertych A, et al (2009). Automated quantification of DNA demethylation effects in cells via 3D mapping of nuclear signatures and population homogeneity assessment. Cytometry A 75, 569-583.
[8] Kullback S, Leibler R A (1951). On information and sufficiency. Ann Math Stat 22, 79-86.

The claims of the invention are:

1. A nondestructive in situ method that entails the quantitative measurement of cytosine methylation (load and spatial distribution) in single cell nuclei, for the characterization of cells and tissues for various applications (including but not restricted to basic research, clinical diagnostics and prognostics, pharmacology and toxicology, and molecular and cell-based therapy), comprising steps of:
a) treatment means for simultaneous fluorescent delineation of methylcytosine and global DNA;
b) procurement means for high-resolution imaging of treated cells and tissues;
c) analyzing means for spatial codistribution of fluorescence signals of nuclear methylcytosine (MeC) and global DNA (gDNA) in imaged cells and tissues.

2. The method of claim 1, which measures the structural similarity between cells based on the MeC load and the MeC/gDNA codistribution pattern.

3. The method of claim 1, which measures the homogeneity of a cell population based on the degree of structural similarities described in claim 2.
